(19) United States
(12) Patent Application Publication
(10) Pub. No.: US 2016/0345926 A1
(43) Pub. Date: Dec. 1, 2016

(54) ACQUISITION OF PROJECTION DATA FOR MOTION-CORRECTED COMPUTED TOMOGRAPHY IMAGES

(71) Applicant: Samsung Electronics, Co. Ltd.

(72) Inventors: Sandeep Dutta, Celebration, FL (US); Ajay Narayanan, Suwon-si (KR)

(21) Appl. No.: 14/964,339

(22) Filed: Dec. 9, 2015

Related U.S. Application Data
(60) Provisional application No. 62/167,126, filed on May 27, 2015.

(51) Int. Cl. A61B 6/00; A61B 6/03

(52) U.S. Cl. CPC A61B 6/5264; A61B 6/461; A61B 6/4435; A61B 6/54; A61B 6/032; A61B 6/503

(57) ABSTRACT

Embodiments are adapted for acquiring projection data from a computed tomography imaging system to form motion-corrected images of cyclically moving imaging subjects. Embodiments determine a start time and time duration of a data record with sufficient data for forming a motion-corrected image from the data record, without screening data records or images for motion artifacts. Some embodiments acquire data asynchronously with respect to the cyclical motion of the imaging subject, without using a signal such as an EKG to select a period of low subject motion for imaging. Other embodiments use a pulse signal synchronized to the imaging subject's motions to reduce an amount of data presented for image reconstruction, or to target a specific phase of the imaging subject's cyclical motion.

### CROSS REFERENCE

This application claims priority to U.S. Provisional Patent Application No. 62/167,126 filed May 27, 2015, titled “System and Method for Motion Free Computed Tomography Images”, incorporated herein by reference in its entirety.

### TECHNICAL FIELD

An embodiment relates in general to imaging by computed tomography, and more specifically to acquisition of projection data for generating an image without motion artifacts for a moving imaging subject.

### BACKGROUND

Computed Tomography (CT) images may be created by combining X-ray images taken while rotating a gantry holding an x-ray source and detector around an imaging subject. For some CT imaging systems, the best CT images may be produced when all parts of the imaging subject are stationary with respect to the imaging system. Motions by the imaging subject may cause degradation of a CT image formed from the individual X-ray images. CT image degradations from subject motion may be referred to as motion artifacts. Examples of motion artifacts from subject motion include loss of image contrast, loss of image resolution, blurring of some or all of an image, and other undesirable effects.

CT imaging has many important medical applications but has proven challenging when imaging parts of a body that undergo movement. A motion artifact in a CT image may obscure a clinically important aspect of the image or may be mistaken for an indication of the condition of the imaging subject. Diagnostic accuracy may suffer as a result of the presence of motion artifacts in CT images. For medical imaging, examples of image subject motions which may cause motion artifacts include motions of heart muscles, coronary vessels, the lungs and other parts of the chest cavity moving in response to breathing, opening and closing of valves and orifices in the circulatory and digestive systems, peristaltic waves, and other voluntary and involuntary motions of the imaging subject. For industrial imaging, examples of image subject motions which may cause motion artifacts include rotating and reciprocating parts of pumps, compressors, generators, motors, engines, linear and rotary actuators, solenoids, switches, relays, and other devices having moving parts.

Techniques and instruments have been developed to improve CT imaging of subjects having moving parts. For example, cardiac CT imaging may use an electrocardiogram (ECG or EKG) signal to determine the time interval during a heartbeat cycle when view data for a CT image will be acquired. The EKG signal may be used to predict the portion of a heartbeat when heart motions are at or near a minimum, as may occur for example during diastole for low heart rates and systole for high heart rates, where a heart rate is an expression of the number of complete heart cycles per unit time. Techniques have been developed to acquire view data for forming a CT image from a single heart cycle and from multiple heart cycles. For example, view data may be collected throughout the duration of a complete heart cycle and then CT images may be made retrospectively at selected times during the heart cycle to find the time relative to the start of a cycle, also referred to as a phase of a heart cycle, at which the best image for diagnostic purposes can be made.

Several problems are associated with using an EKG signal to control acquisition and selection of CT images. Placement of sensors and connecting wires to detect EKG signals that can be compared to diagnostic references requires skilled, experience staff who may not be available at the time a CT image is to be made. Electrical noise picked up by the EKG sensors or wires may interfere with interpretation of EKG signals. The imaging subject's heart rate may be so high that a sufficiently stable period for CT imaging cannot be located, possibly requiring the administration of drugs to reduce heart rate. Using an EKG to control CT imaging may therefore interfere with workflow in a CT imaging facility or increase the cost of CT imaging. The person responsible for selecting and interpreting CT images must know which EKG sensor, and which portion of an EKG signal from a selected sensor, may produce a CT image of the selected phase that has the fewest number of motion artifacts, and must be able to distinguish between a motion artifact and an actual medical condition of the imaging subject.

Improper visualization from motion artifacts may lead to errors in identification of pathology and possibly to misdiagnosis. For example, a physician may need to closely and carefully examine many CT images at different phases of a heart cycle to find an image that is sufficiently free of motion artifacts for accurate diagnosis. The skill of the operator of the CT instrument may affect the quality of CT images and the usefulness of the images for diagnostic purposes. The difficulty is increased when the clinically relevant portions of an imaging subject are small in size, for example when looking for the presence of plaque or blockage in the circulatory system. The time and experience needed required of clinical staff to create and sort through CT images has impeded the use of CT imaging and increased the cost of CT imaging.

Algorithms have been developed to perform pre-processing of view data in the projection or image domain to find a phase of least motion for capturing images of the heart and vessels. However, these algorithms are unable to guarantee that the resulting CT images will be free from motion artifacts, for reasons including the variability of heart rate in some patients, premature heart beats, and errors in feature detection algorithms caused by noise and low image contrast.

Post-acquisition image processing methods have been introduced to reduce motion artifacts from images of coronary vessels. These methods may depend on accurate identification of a coronary vessel tree from image data to detect and compensate for motions of the vessels, but may fail to eliminate the effects of large imaging subject motions and high noise levels from CT images.

Previously known methods have been unable to produce high-resolution CT images of rapidly moving imaging subjects without using complex gating signals such as an EKG. Selection of the wrong EKG lead, or selecting the wrong part of an EKG signal on a correctly chosen lead, may result in a CT image with motion artifacts that obscure or misrepresent diagnostically important aspects of the imaging subject. Producing high-contrast, high-resolution CT images that are free from motion artifacts, without depending on the skill and experience of an imaging system operator to interpret the physiological and imaging implications of a gating signal, are problems that have remained unsolved by previously known methods.

### SUMMARY

An example of a method embodiment includes determining a time duration t_{R} of a data record to reconstruct exactly one motion-corrected computed tomography (CT) image of an imaging subject having a moving part; rotating a gantry holding a detector and a source through a full revolution; and while rotating the gantry, capturing a sequence of views of the imaging subject for the time duration t_{R}. The example of a method embodiment further includes saving the sequence of views in the data record; forming a motion-corrected image of the imaging subject by partial angle reconstruction of the sequence of views from the data record; and displaying the motion-corrected CT image.

An embodiment may optionally include acquiring view data asynchronously to a heart cycle. Alternatively, an embodiment may optionally include acquiring view data synchronously with a pulse signal representative of a heart rate.

### BRIEF DESCRIPTION OF THE DRAWINGS

Accompanying drawings show one or more embodiments; however, the accompanying drawings should not be taken to limit the invention to only the embodiments shown. Various aspects and advantages will become apparent upon review of the following detailed description and upon reference to the drawings in which:

FIG. 1 is a block diagram of an exemplary method embodiment.

FIG. 2 is a schematic representation of examples of parts of a computed tomography (CT) imaging system in accord with an embodiment.

FIG. 3 shows an example of a rotation of the gantry from FIG. 2 from a first gantry position to a second gantry position, an example of a beam from a source to a detector having a fan angle θ_{F}, an example of a gantry rotation angle θ_{G} greater than 180° and less than 360°, and an example of a rate of gantry rotation R_{G}.

FIG. 4 is a schematic diagram of an exemplary apparatus embodiment, showing more parts of the example of a CT imaging system from FIGS. 3-4, and further illustrating an example of a motion-corrected CT image 158 representing an example of an imaging subject 300 having a moving object 302.

FIG. 5 is a timing diagram showing an example of timing relationships between data records acquired asynchronously relative to a heart cycle represented by an EKG signal. An EKG signal is not required by any of the embodiments disclosed herein to produce a motion-corrected CT image.

FIG. 6 is a timing diagram of an example of a pulse signal synchronous with an EKG signal. The pulse signal may optionally be used to reduce an amount of data collected and processed by an embodiment to produce a motion-corrected CT image.

FIG. 7 is an example of an alternative embodiment for synchronous data acquisition, showing an example of parameters for collecting view data centered on a target phase to produce a motion-corrected CT image at the target phase.

FIG. 8 is a block diagram of an example of a central processing unit 142 in accord with an embodiment.

FIG. 9 is a block diagram of an example of a computer program product embodiment.

### DESCRIPTION

An exemplary embodiment improves acquisition of view data for computed tomography (CT) images by collecting a data record, and in some embodiments exactly one data record, having sufficient information to produce a motion-corrected image. A data record in accord with an embodiment may be collected asynchronously with respect to a cyclical motion of an imaging subject. A data record collected asynchronously with respect to the motions of the imaging subject may be used to generate motion-corrected CT images without using or referencing any signal representing motions of the imaging subject, such as an EKG signal or a timing pulse measured from the imaging subject. However, when a signal having components which are synchronous with motions of the imaging subject is available, embodiments may use the synchronization signal to reduce an amount of data presented to an image reconstruction system for formation of a motion-corrected CT image, and may optionally use the synchronization signal to target image acquisition at a selected time during a cyclical motion of the imaging subject.

Embodiments are advantageous for collecting projection data, also referred to as view data, to be combined into a data record submitted to an image reconstruction system for forming motion-compensated CT images. In contrast to previously known methods for CT imaging of moving imaging subjects, embodiments eliminate the screening of multiple CT images or the associated data records, either by a person or by a screening algorithm, to identify an image with motion artifacts that are too small to interfere with diagnostic accuracy.

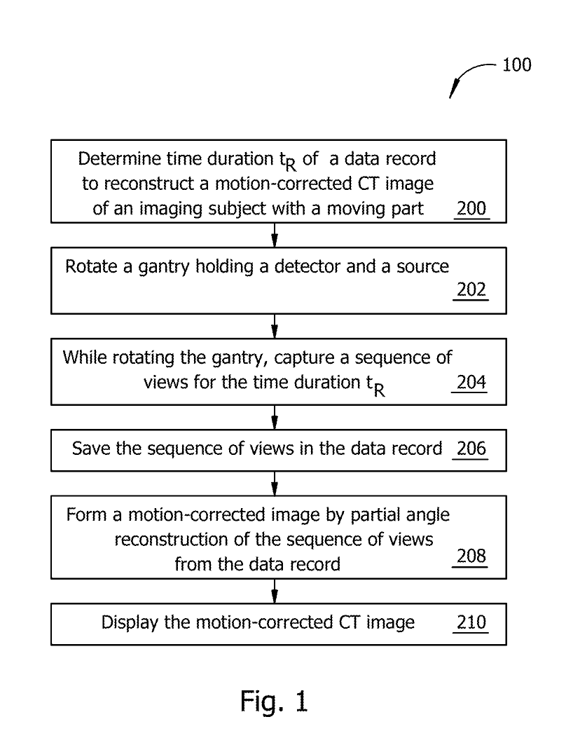

FIG. 1 illustrates some of the operations performed by an exemplary method embodiment. The example of a method embodiment begins at block 200 with determining a time duration t_{R} of a data record having sufficient information for an image reconstruction system to reconstruct a motion-corrected CT image of an imaging subject having a moving part. Next, at block 202, a gantry in a CT imaging system is rotated to position an X-ray source and an X-ray imaging detector at incremental angular intervals around the imaging subject. A view of the imaging subject is captured at each incremental position during the gantry's rotation to create a sequence of views as suggested in block 204. Each view may be represented by digital data output from the detector 104. The sequence of views is captured for the time duration t_{R}, which may be less than the time for a rotation of the gantry through 360°, but which will preferably be longer than the time for the gantry to rotate through 180° to enable the collection of conjugate views for partial angle reconstruction, a technique for forming motion-corrected images from conjugate image pairs. Each view in the sequence of views collected during the time duration t_{R} is saved in the data record at block 206. At block 208, an image reconstruction system reads the sequence of views in the data record and reconstructs a motion-corrected CT image using partial angle reconstruction. At block 208, the motion-corrected CT image is presented on a display. It is an important property of the disclosed embodiments that only one data record needs to be presented to the image reconstruction system to generate a motion-corrected CT image, and the associated motion-corrected CT image is free of motion artifacts that could interfere with accurate diagnosis of the imaging subject.

The time duration of the one data record depends on whether the acquisition is performed synchronously or asynchronously relative to cyclical motions of the imaging subject, as will be discussed in more detail below. Synchronous acquisition refers to data records having a start time deliberately aligned in time with a specific event in a cyclical motion of the imaging subject, for example a selected heart phase or the time of a synchronization pulse. Asynchronous acquisition refers to a data record that is not deliberately time-aligned with an event in the cyclical motion of the imaging subject, and in which events occurring during the cyclical motion may appear at any time during the acquisition of the data record.

For discussion purposes herein, a cyclical motion is a motion which repeats at approximately regular intervals. A period of the cyclical motion is a time duration from an event in one cycle to the corresponding event in the next cycle. For the embodiments disclosed herein, it is not necessary for the cyclical motion to repeat with perfect uniformity. An image free from motion artifacts may still be reconstructed even when one period of cyclical motion does not exactly match another period of cyclical motion. This contrasts with some previously known methods which may collect data for reconstruction of one image by combining image data from more than one period of cyclical motion of the imaging subject, where any variation in subject motion from one period to another may lead to image degradation. Embodiments preferably collect a minimum amount of data needed to reconstruct exactly one motion-corrected CT image and preferably avoid collecting more than the minimum amount of data needed for image reconstruction. A motion-corrected CT image in accord with an embodiment is a CT image formed by partial angle reconstruction in which any motion artifacts that may be present are too small to affect the accuracy of a diagnosis of the imaging subject based on the reconstructed CT image.

CT images in accord with an embodiment are preferably formed by partial angle reconstruction. Partial angle reconstruction is capable of forming high-resolution CT images in which the subject motion is averaged over a much shorter time period than other methods of CT imaging. For example, at a heart rate of 75 beats per minute, the duration of one heart cycle is 800 milliseconds (ms). For a gantry rotation rate of 240 revolutions per minute, partial angle reconstruction is capable of producing a CT image with subject motion averaged over an interval of about 175 microseconds, a duration over which heart motion is insignificant for most diagnostic imaging purposes, except at the most rapid portion of a heart cycle represented on an EKG by an interval including the peak R in a QRS complex. Some embodiments operate to provide sufficient data for reconstruction of a motion-corrected image even when the QRS complex occurs during collection of view data. Other embodiments collect view data away from the relatively short portion of a heart cycle associated with maximum heart motion. In contrast to partial angle reconstruction techniques used by an embodiment, other previously known CT imaging methods may average subject motion over an interval of about 60 ms, long enough for many of the heart motions that occur during a heart cycle to cause motion artifacts that are large enough to interfere with diagnostic interpretation of CT images.

To perform partial angle reconstruction, a CT imaging system acquires a set of views corresponding to the fan angle of an X-ray beam and another set of views with the gantry rotated 180° from the first set of views. The two sets of views represent conjugate views of the imaging subject. The conjugate views are compared to one another to determine which parts of the imaging subject may have moved from one view to another, and a motion correction estimate is formed based on the analysis of the sequence of views. The motion correction estimate is applied to the views to compensate for subject motion, and the CT image is reconstructed from motion-compensated data to form a motion-corrected image. At present, partial angle reconstruction is capable of compensating for heart motions everywhere in a heart cycle but very close to the peak R of a QRS complex. Heart motions during other parts of a heart cycle are sufficiently long compared to the temporal resolution of the partial angle reconstruction technique that motions away from the peak of the QRS complex for a normal sinus rhythm do not cause motion artifacts that could affect diagnostic accuracy. Motions of other organs, for example respiratory motions, are substantially slower than the fastest heart motions than can be imaged and may therefore be imaged accurately without motion artifacts by partial angle reconstruction.

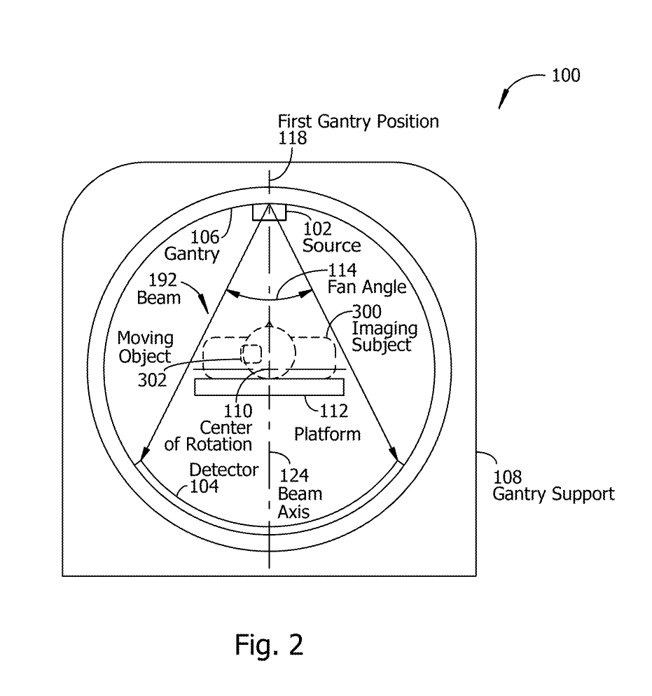

FIG. 2 shows an example of a CT imaging apparatus 100 in accord with an embodiment. In the example of FIG. 2, a gantry 106 is rotationally coupled to a gantry support 108. An X-ray source 102 and an X-ray imaging detector 104 are attached to and rotate together with the gantry 106. A beam of X-rays 192 traverses from the source 102 to the detector 104 along a beam axis 124 which may pass through a center of rotation 110. The portion of the beam which may be received by the detector diverges at a fan angle 114. An imaging subject 300 may rest on a platform 112 within the perimeter of the gantry 106. The imaging subject 300 may include a part which moves relative to other parts of the subject, represented in the figure by a moving object 302. The imaging subject 300 and the moving object 302 are not included in an embodiment.

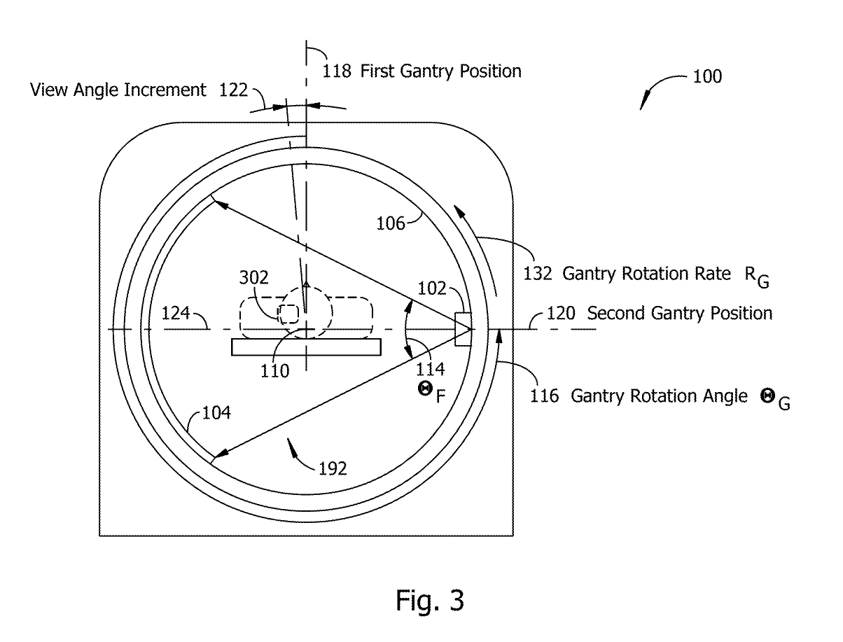

FIG. 3 continues the example of FIG. 2, showing the gantry 106 rotated from a first gantry position 118 to a second gantry position 120 through a gantry rotation angle θ_{G} 116. The beam axis 124 rotates with the gantry 106. The gantry may be rotated at a rate of rotation R_{G}. A view angle increment, that is, an angular separation between adjacent views of the moving object 302, may be determined by details of the detector 104, for example the number and spacing of sensing elements distributed across the fan angle θ_{F} 114. Some CT imaging systems use a detector that is large enough to image the entire width of an imaging subject at one time. Other CT imaging systems reconstruct a complete image by rotating the gantry to capture sub-images, each sub-image capturing a portion of the imaging subject, and combining the sub-images. Partial angle reconstruction may be used with either of these reconstruction methods.

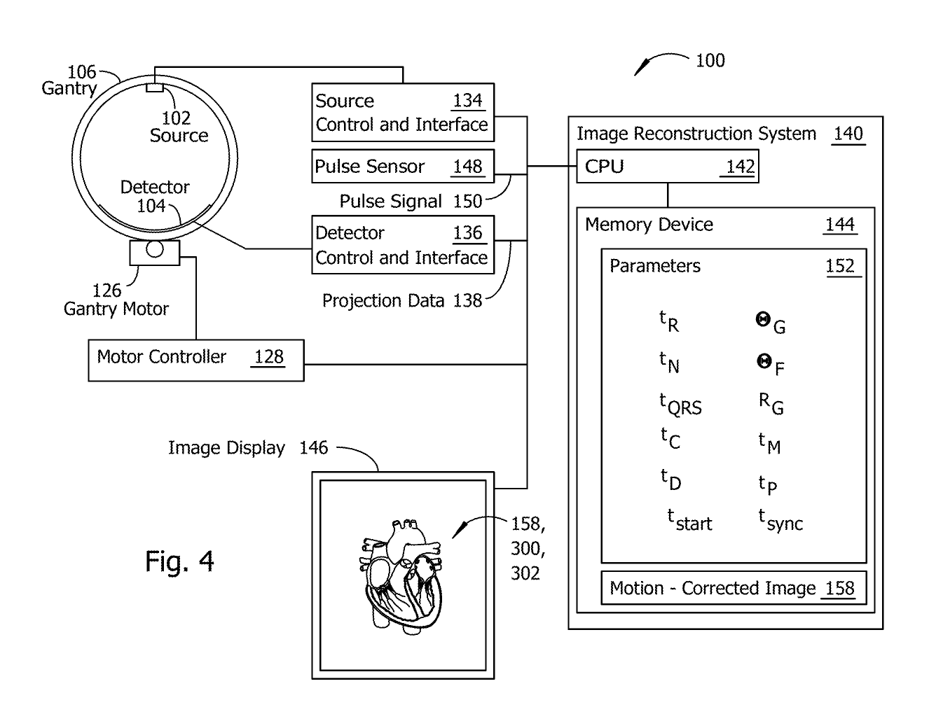

FIG. 4 shows examples of some additional details of a CT imaging system in accord with an exemplary embodiment to form a motion-corrected CT image. In the example of FIG. 4, the gantry 106 is driven in rotation by a gantry motor 126 controlled by a motor controller 128. The motor controller 128 responds to commands from an image reconstruction system 140 to rotate the gantry 106 at a selected rotation rate R_{G}, and optionally to report a gantry rotation angle θ_{G} to a central processing unit (CPU) 142 in the image reconstruction system. The CPU 142 is connected for data communication with a memory device 144 for storing projection data 138, also revered to as view data, parameters 152 including gantry control parameters and data acquisition parameters, and a motion-corrected image 158 which may be displayed on an image display 146.

The image reconstruction system 140 may activate output from the source 102 through a source control and interface 134. Projection data 138 from the detector 104 may be received by the CPU 142 from a detector control and interface 136. In some embodiments, a pulse signal 150 synchronized with a cyclical motion of the moving object in the imaging subject may be detected by an optional pulse sensor 148 and communicated to the CPU 142. Examples of a pulse sensor 148 for cardiac imaging include, but are not limited to, a pulse Doppler probe, an acoustic sensor, a pressure sensor, and a pulse oximetry probe, any of which may output a pulse signal useful for synchronous acquisition of view data.

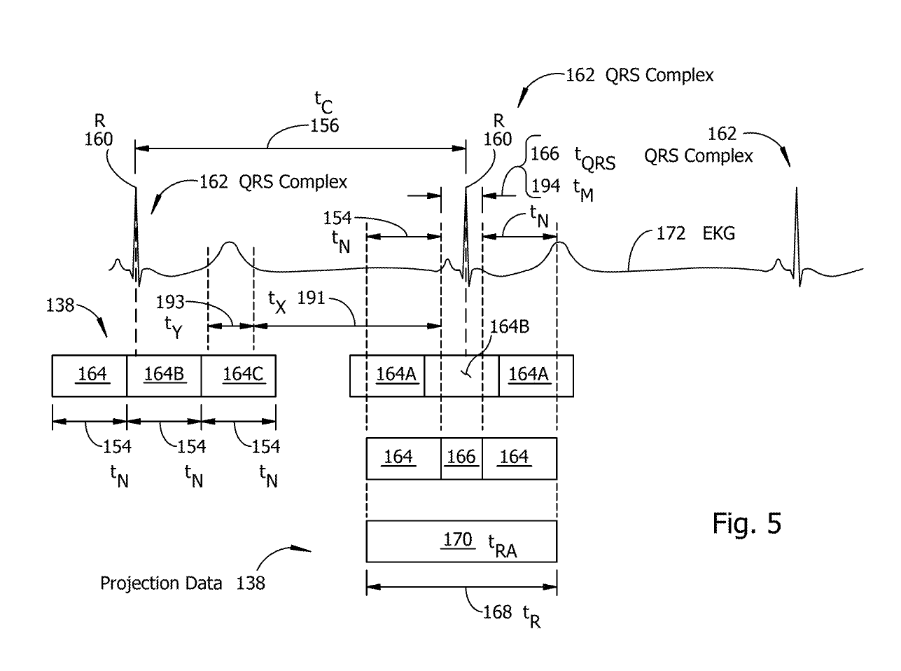

FIG. 5 illustrates an example in which an embodiment performs acquisition of view data asynchronously with respect to a cyclical motion of the imaging subject. Cyclical motion of an imaging subject is represented in the examples of the figures by an EKG signal 172. The EKG signal 172 is not required for operation of an embodiment and is not used by the exemplary embodiment to select a portion of a heart cycle suitable for forming a motion-corrected CT image.

To produce a conjugate image pair for partial angle reconstruction, the gantry may be swept through a gantry rotation angle θ_{G} determined by equation (1):

θ_{G}=180°+θ_{F}+θ (1)

where θ_{F} is the fan angle of the beam, θ is an optional incremental angle used by some embodiments, and 0°<θ_{G}<360°. The time required for the gantry to traverse θ_{G} at a specified gantry rotation rate R_{G} determines the minimum time duration t_{N} 154 of a data record 164 having sufficient data to form a motion-corrected CT image by partial angle reconstruction.

As noted earlier, imaging by an embodiment preferably avoids the period of maximum heart motion near the peak R 160 of the QRS complex 162 in the EKG signal example 172. When a data record 164 is acquired asynchronously, at some times during the cycle the data record may by chance be collected concurrently with the maximum heart motion and at other times may by chance be collected when the heart motion is within the motion correction capabilities of partial angle reconstruction. For the examples of data records 164 in FIG. 5, each of duration t_{N} 154, any of the examples of a data record 164A have been collected away from the period of maximum heart motion at peak R 160, while other examples of data records 164B have been collected concurrently with the QRS complex 162 and may therefore result in motion artifacts in a CT image. However, in contrast to other CT imaging methods, an embodiment may present exactly one data record for image reconstruction by asynchronously acquiring a data record of duration t_{RA}, 170 according to equation (2):

t_{RA}=2×t_{N}+t_{M} (2)

where t_{M} 194 is the duration of a time interval during which subject motion may not be well-corrected by partial angle reconstruction. An embodiment performs asynchronous acquisition by rotating the gantry at a specified rate of rotation R_{G} and determining the time duration t_{N} for the gantry to rotate through the gantry rotation angle θ_{G} at the specified rate of rotation R_{G}.

Acquiring view data for the time duration t_{RA} may be performed without concurrent reference to an EKG signal or any other timing signal related to the cyclical motion of the imaging subject, for example a signal representative of a heartbeat. For cardiac imaging, the interval t_{M} 194 may correspond to the duration of the QRS complex 162, t_{QRS} 166. A data record 170 of duration t_{RA} 168 will always contain projection data 138 in a continuous block with time duration t_{N} 154 to guarantee that the image reconstruction system can form a motion-corrected CT image, no matter when the interval of maximum subject motion t_{M} 194 occurs relative to the acquisition of projection data. Only the one data record 170 of duration t_{RA} need be processed by the image reconstruction system to form a motion-corrected image 158, without screening or sorting of multiple CT images or data records.

Heart motions sufficiently large to cause motion artifacts in previously known CT imaging systems may occur at phases of the heart cycle other than the QRS complex. For example, heart motion may be sufficient to cause undesirable motion artifacts in a CT image formed by previously known CT systems from projection data 164C that includes data from the interval t_{Y} 193 in FIG. 5. Operators of previous CT imaging systems may reference an EKG signal during CT imaging to target the time interval t_{X} 191 for cardiac imaging, where the interval t_{X} on the EKG may correspond to an interval of reduced heart motion. However, as heart rate increases, the interval t_{X} may decrease in duration, making it more difficult to form an image of the heart free from motion artifacts. Or, an EKG signal may include signal components such as electrical noise or deviations from normal heartbeats which make it difficult to identify interval t_{X} 191. An operator of a CT system who is not experienced with interpreting EKG signals may acquire CT images with motion artifacts. The embodiments disclosed herein avoid these complications by forming motion-corrected CT images without requiring an EKG signal to acquire projection data suitable for reconstruction of motion-corrected CT images. In contrast to other CT systems, data collected during record 164C in the example of FIG. 5 will result in a motion-corrected CT image when the record is processed in accord with an embodiment.

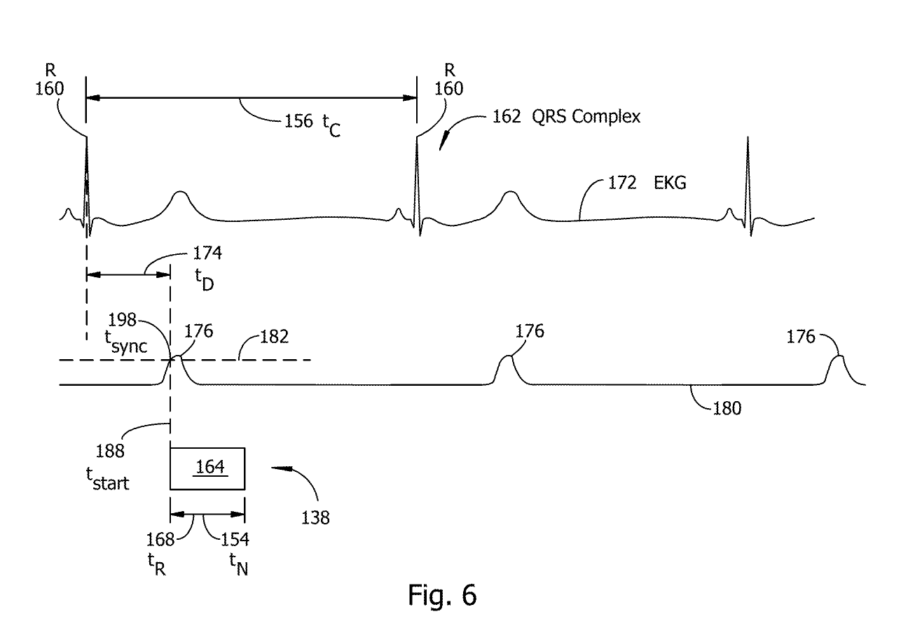

Some embodiments may optionally coordinate acquisition of view data with a synchronization signal to reduce an amount of view data acquired for CT image reconstruction. In the example of FIG. 6, a synchronization signal 180 includes a periodic component represented in the figure by a pulse 176 having a synchronous time relationship to the cyclical motion of the imaging subject, where the cyclical motion is represented in FIG. 6 by the EKG signal 172. For example, the pulse may have a known, predicted, or estimated value for a time delay t_{D} 174 from a reference point in the cyclical motion, perhaps peak R 160 in a QRS complex 162. As for other embodiments, the EKG 172 is shown in the example of FIG. 6 as a representation of cyclical heart motions and is not required for forming motion-corrected images. The delay interval t_{D} 174 may be measured, for example, as the time t_{sync} 198 at which the amplitude of the pulse 176 passes through a detection threshold 182. The detection time t_{sync} may alternatively be determined by detecting the time of the peak of the pulse or by other methods. When the time delay t_{D} 174 is known to be large enough to separate a data record 164 from the period of maximum heart motion, the synchronization signal 180 may trigger the acquisition of view data 138 for the duration t_{N} 154 and present the data record 164 to the image reconstruction system for formation of a motion-corrected CT image. The synchronization signal may optionally be used to specify a gantry rotation rate R_{G} so that at least one complete data record 164 is collected during the period t_{C} 156 of a heartbeat.

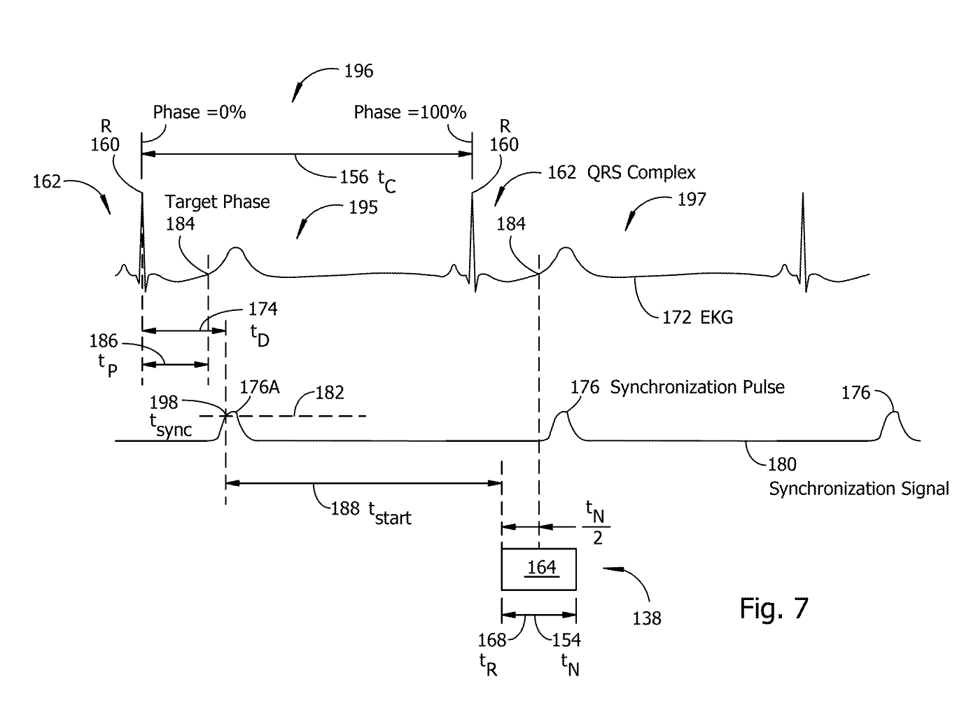

When the detected time t_{sync} 198 of the synchronization pulse 176 on the synchronization signal 180 occurs too close to the QRS complex 162, or when it is desirable to target a specific heart phase for imaging, an embodiment may perform synchronous data acquisition in accord with the example of FIG. 7. As for other embodiments, the example of an EKG signal 172 represents phases of cyclical heart motion and is not required to produce a motion-corrected CT image. As suggested in FIG. 7, an embodiment may delay acquisition of view data from the heart cycle 195 during which the synchronization pulse 176A is detected at time t_{sync} 198 until the next sequential heart cycle 197, then collect one data record 164 of duration t_{N} to record the minimum amount of projection data 138 needed for reconstruction of one motion-corrected image.

FIG. 7 shows an example synchronous data acquisition using a pulse 176A on the synchronization signal 180 that has a delay time t_{D} 174 from a known part of the cyclical motion of the imaging subject, for example the peak R 160 of the QRS complex 162. The time delay t_{D} may alternatively be defined with respect to another repeated feature on an EKG, or a repeated feature on another signal representative of cyclical motion of the imaging subject. An interval t_{C} 186 may represent a time duration from peak R 160 to the desired target phase 184 for imaging. The target phase for imaging may be expressed, for example, as a percentage of the period t_{C} 156 of one complete heartbeat cycle 196 extending from phase=0% at a first peak R 160 to phase=100% at the next sequential R peak (the “R-R interval”) on the EKG 172. To collect sufficient view data for reconstruction of one motion-corrected image centered at the selected heart phase 184, acquisition of view data preferably begins after a time interval tₛₜₐᵣₜ 188 following the time of detection t_{sync} 198 of the pulse 176, where the length of the interval tₛₜₐᵣₜ may be determined from equation (3):

tₛₜₐᵣₜ=|t_{C}−t_{D}|+(t_{P}−(t_{N}/2) (3)

and where |t_{C}−t_{D}| represents the absolute value of the difference of t_{C} and t_{D}. Collection of view data continues for a time duration of t_{N} 154 and the resulting data record 164 is submitted to the image reconstruction system for formation of one motion-corrected image centered at the selected heart phase 184.

Although embodiments do not require an EKG signal to form motion-corrected CT images, if an EKG signal is available it may be used as the synchronization signal 180. For example, the R peak of a QRS complex may be used to image at a specified target phase, according the operations performed with respect to FIG. 7. Even when an EKG signal is used by an embodiment, only one data record needs to be acquired, and no screening of data records or CT images needs to occur to find an image free of motion artifacts.

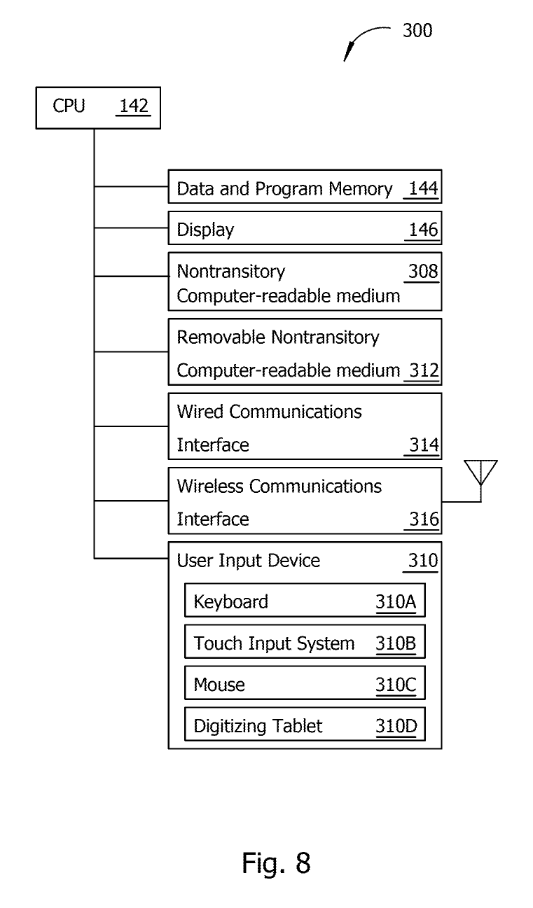

FIG. 8 shows some details of an embodiment including a computer apparatus adapted to execute software instructions implementing a method embodiment, for example the example of a method shown in FIG. 1. The computer apparatus 300 in the example of FIG. 8 includes a central processing unit (CPU) 142 implemented as a hardware device. Hardware in accord with an apparatus embodiment 300 may be implemented as electrically interconnected semiconductor devices, possibly in the form of at least one integrated circuit. The CPU 142 is electrically connected for exchange of data and program instructions with a memory device, for example a data and program memory 144 implemented in hardware. The CPU 142 further communicates with a display device 146, for example a liquid crystal display, electroluminescent display, vacuum fluorescent display, light emitting diode (LED) display, cathode ray tube (CRT) display, or a plasma display, a user input device 310, and a storage device including computer-readable media, for example a computer-readable medium 312 and optionally a removable computer-readable medium 312. Examples of a user input device 310 include, but are not limited to, a keyboard 310A, a touch input system 301B, a mouse 310C, a trackball, a joystick, a digitizing tablet 310D, a tablet computer 310E, and a cellular telephone with a graphical user interface and Internet browsing capability, also referred to as a smart phone 310F. The CPU may optionally send and receive calculation results, input data, and program instructions through an optional wired communications interface 314, an optional wireless communications interface 316, or possibly through both wired and wireless communications interfaces (314, 316). Examples of wired communications interfaces include, but are not limited to, Ethernet, twisted pair, a parallel computer interface, an optical fiber network interface, and a serial computer interface. Examples of wireless communications interfaces include, but are not limited to, Bluetooth™, a cellular telephone network, IrDA, and Wi-Fi.

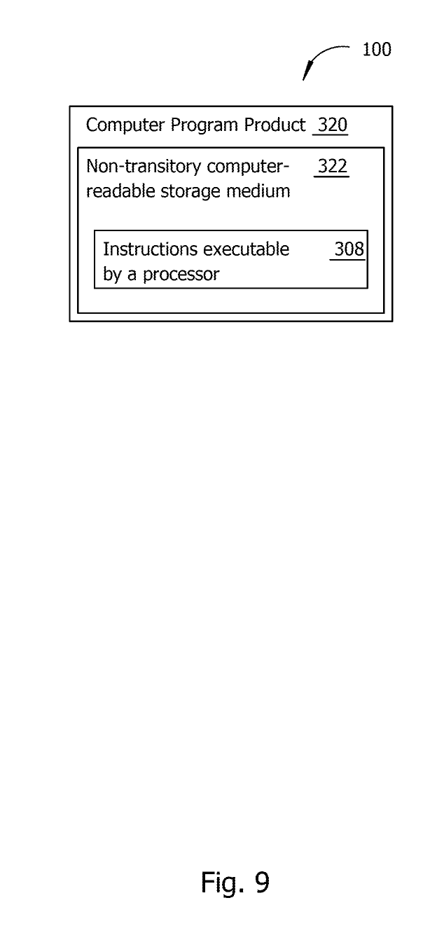

FIG. 9 shows an example of an apparatus embodiment comprising a computer program product 320. The computer program product 320 may be implemented on a non-transitory computer-readable storage medium 322 and may include instructions executable by a processor 308, for example instructions for implementing the example of a method in FIG. 1.

Some of the operations described herein may be performed in a different order than implied by the sequence of blocks in FIG. 1. Such variations are considered to be within the scope of the disclosed embodiments.

Unless expressly stated otherwise herein, ordinary terms have their corresponding ordinary meanings within the respective contexts of their presentations, and ordinary terms of art have their corresponding regular meanings.

What is claimed is:

1. A method comprising:
determining a time duration t_{R} of a data record to reconstruct exactly one motion-corrected computed tomography (CT) image of an imaging subject having a moving part;
rotating a gantry holding a detector and a source;
while rotating the gantry, capturing a sequence of views of the imaging subject for the time duration t_{R};
saving the sequence of views in the data record;
forming a motion-corrected image of the imaging subject by partial angle reconstruction of the sequence of views from the data record; and
displaying the motion-corrected CT image.

2. The method of claim 1, wherein capturing the sequence of views occurs over a gantry rotation angle θ_{G} greater than one hundred eight degrees (180°) and less than three hundred sixty degrees)(360°, 180°<θ_{G}<360°.

3. The method of claim 1, further comprising capturing the sequence of views asynchronously relative to a cyclical motion of the imaging subject.

4. The method of claim 3, further comprising determining a gantry rotation angle θ_{G} by adding a magnitude of a fan angle θ_{F} to one hundred eighty degrees (180°), according to θ_{G}=θ_{F}+180°.

5. The method of claim 4, further comprising:
rotating the gantry at a specified rate of rotation R_{G}; and
determining a time duration t_{N} for the gantry to rotate through the gantry rotation angle θ_{G} at the specified rate of rotation R_{G}.

6. The method of claim 4, further comprising:
determining a time duration t_{M} in the cyclical motion of the imaging subject in which a motion artifact may appear in the corresponding motion-corrected image; and
determining the time duration t_{R} by multiplying the time duration t_{N} by 2 and adding the time duration t_{M}, according to t_{R}=(2×t_{N})+t_{M}.

7. The method of claim 6, wherein a time duration t_{QRS} of a QRS complex is selected as a value for the time duration t_{M}.

8. The method of claim 6, wherein the time duration t_{R} is selected to include at least one continuous interval of duration t_{N} separate from an interval of duration t_{M}.

9. The method of claim 1, further comprising selecting a rate of rotation R_{G} for the gantry such that a period of one revolution of the gantry through three hundred sixty degrees (360°) is less than a period t_{C} of one complete heartbeat.

10. The method of claim 1, wherein the sequence of views is captured without detecting an electrical signal having a periodic component synchronized in time with a cyclical motion of the imaging subject.

11. The method of claim 1, further comprising selecting a beating heart as the imaging subject having a moving part.

12. The method of claim 1, wherein determining the time duration t_{R} of the data record comprises:
determining a gantry rotation angle θ_{G} by adding a magnitude of a fan angle θ_{F} to one hundred eighty degrees (180°), according to θ_{G}=θ_{F}+180;
rotating the gantry at a specified rate of rotation R_{G};
determining a time duration t_{N} for the gantry to rotate through the gantry rotation angle θ_{G} at the specified rate of rotation R_{G}; and
assigning the time duration t_{N} to the time duration t_{R} of the data record.

13. The method of claim 12, further comprising:
detecting a time of occurrence of a periodic component of a synchronization signal;
beginning at the time of occurrence of the periodic component of the synchronization signal, capturing the sequence of views for the time duration t_{R} of the data record.

14. The method of claim 12, wherein the synchronization signal is a heart rate signal from a pulse oximeter.

15. The method of claim 12, wherein the synchronization signal is a pulse component of an electrocardiogram.

16. The method of claim 12, further comprising selecting a target phase for imaging in a heartbeat cycle, wherein the synchronization signal comprises a periodic pulse occurring a same number of times in each sequential heartbeat cycle.

17. The method of claim 16, further comprising:
assigning a value for a time delay t_{D} from a QRS complex to the detected time of occurrence of the synchronization signal;
determining a time interval t_{P} from the start of a heartbeat cycle to the target phase;
assigning a value to a period t_{C} of one complete heartbeat cycle; and
acquiring the sequence of views of the imaging subject beginning at a time interval equal to (t_{N}/2) before the target phase in the next sequential heartbeat cycle following the time of detection of the synchronization signal.

18. The method of claim 17, further comprising:
from the detected time of occurrence of the synchronization signal, delaying acquiring the sequence of views by a time interval tₛₜₐᵣₜ determined by adding the absolute value of the difference between the period of one heartbeat cycle t_{C} and the time delay t_{D} to the difference between time interval t_{P} and half of the time interval t_{N}, in accord with tₛₜₐᵣₜ=|t_{C}−t_{D}|+(t_{P}−(t_{N}/2); and
acquiring a data record of duration t_{N}.

19. An apparatus, comprising:
a gantry comprising a source and a detector;
a motor controller connected to said gantry, said motor controller adapted to rotate said gantry;
a processor connected to said motor controller;
a memory coupled to said processor, said memory including instructions executable by the processor to:
determine a time duration t_{R} of a data record to reconstruct exactly one motion-corrected computed tomography (CT) image of an imaging subject having a moving part;
rotate a gantry holding a detector and a source;
while rotating the gantry, capturing a sequence of views of the imaging subject for the time duration t_{R};
save the sequence of views in the data record; and
form a motion-corrected image of the imaging subject by partial angle reconstruction of the sequence of views from the data record.

20. A computer program product, comprising a non-transitory computer-readable storage medium including instructions executable by a processor comprising:
determining a time duration t_{R} of a data record to reconstruct exactly one motion-corrected computed tomography (CT) image of an imaging subject having a moving part;
rotating a gantry holding a detector and a source;
while rotating the gantry, capturing a sequence of views of the imaging subject for the time duration t_{R};
saving the sequence of views in the data record;
forming a motion-corrected image of the imaging subject by partial angle reconstruction of the sequence of views from the data record; and
displaying the motion-corrected CT image.
